(19) United States
(12) Patent Application Publication
(10) Pub. No.: US 2023/0167411 A1
(43) Pub. Date: Jun. 1, 2023

(54) USE OF CD34 AS A MARKER FOR SINOATRIAL NODE-LIKE PACEMAKER CELLS

(71) Applicant: UNIVERSITY HEALTH NETWORK, Toronto (CA)

(72) Inventors: Stephanie I. Protze, Toronto (CA); Gordon M. Keller, Toronto (CA); Michelle Lohbihler, Toronto (CA); Amos Chun-Hin Adrian Lim, Toronto (CA)

(21) Appl. No.: 17/922,731

(22) PCT Filed: Apr. 30, 2021
(86) PCT No.: PCT/IB2021/053646

§ 371 Date: Nov. 1, 2022

Related U.S. Application Data
(60) Provisional application No. 63/018,824, filed on May 1, 2020.

(51) Int. Cl. C12N 5/077; G01N 33/569; C12Q 1/6881; A61K 35/34

(52) U.S. Cl. CPC C12N 5/0657; G01N 33/56966; C12Q 1/6881; A61K 35/34; C12N 2501/155; C12N 2501/115; C12N 2501/165; C12N 2506/45; C12Q 1/6869

(57) ABSTRACT

The invention relates to the use of CD34 as a cell surface marker to detect sinoatrial node-like pacemaker cells (SANLPCs) in a population of cells and to generate cell preparations highly enriched for SANLPCs. Also provides herein are methods of using SANLPC-enriched cell preparations for cardiac cell therapy.

### CROSS REFERENCE TO RELATED APPLICATION

The present application claims priority from U.S. Provisional Application 63/018,824, filed May 1, 2020. The content of the aforementioned priority application is incorporated herein by reference in its entirety.

### BACKGROUND OF THE INVENTION

The human heartbeat is triggered by electrical impulses that are regulated by two pacemaker tissues: the sinoatrial node (SAN) and the atrioventricular node (AVN). The SAN is located in the posterior wall of the right atrium and serves as the primary pacemaker that initiates the heartbeat. The AVN is located at the base of the right atrium and establishes the electrical connection that sends signals from the atria to the ventricles. SAN and AVN functions can be affected by aging, congenital diseases, or heart surgery, resulting in a slow, irregular heartbeat (bradyarrhythmia) with symptoms ranging from fatigue to syncope. The best treatment currently available for bradyarrhythmia is the implantation of an electronic pacemaker (EPM) that takes over the electrical activation of the heartbeat (Epstein et al., Heart Rhythm (2008) 5:e 1-62).

Anatomically, the SAN can be divided into the SAN head, the SAN tail, and the transition zone (Chandler et al., Circulation (2009) 119:1562-75; Goodyer et al., Circ Res. (2019) 125:379-97; Wiese et al., Circ Res. (2009) 104:388-97). The SAN head contains the core pacemaker cells from which the heartbeat is initiated. These cells derive from TBX18⁺ progenitors in the sinus venosus that develop into the TBX18⁺NKX2-5⁻ pacemaker cells. The lack of NKX2-5 expression distinguishes the SAN head pacemaker cells from all the other cardiomyocytes in the heart, which have been found to express this cardiac transcription factor (Christoffels and Moorman, Circ Arrhythm Electrophysiol. (2009) 2:195-207; Sizarov et al., Circ Arrhythm Electrophysiol. (2011) 4:532-42). The SAN tail originates developmentally from the same TBX18⁺ progenitors but the tail cells upregulate NKX2-5 and downregulate TBX18 (Wiese, supra). The transition zone is located between the SAN and the atrial working myocardium and is comprised of cells having a mixed pacemaker/atrial cardiomyocyte phenotype.

Approximately 21,000 Canadians receive an EPM every year. The number is steadily increasing with the aging population. EPM treatment is associated with a relatively high complication rate (˜16%), mostly caused by thoracic trauma, lead complications, and infections (Cantillon et al., JACC Clin Electrophysiol. (2017) 3:1296-1305; Kirkfeldt et al., Eur Heart J. (2014) 35:1186-94; Udo et al., Heart Rhythm (2012) 9:728-35). Additional downsides of EPM treatment are the lack of autonomic responsiveness and improper electrical excitation of the heart, which can lead to remodeling and eventually heart failure (Sanderson and Yu, Eur Heart J. (2012) 33:816-8; Vatankulu et al., Am J Cardiol. (2009) 103:1280-4). The complication risk for pediatric and adolescent patients is even higher, because EPMs require surgical battery replacements every 5-10 years and periodical surgical refitting of the EPM leads, which lack the ability to adapt to body growth.

These disadvantages represent compelling reasons for the development of biological pacemakers that can be transplanted directly into the SAN site, integrate with the components of the cardiac conduction system that are still intact, and function under the control of the autonomic nervous system. Additionally, biological pacemakers are more likely to adapt to the growth of the heart in pediatric patients. However, progress in the development of biological pacemakers has been hampered by the lack of bona fide human pacemaker cells. Previous studies show that mixed populations of cardiomyocytes differentiated from human pluripotent stem cells (hPSCs) could function as biological pacemakers when transplanted into the pig and guinea pig hearts; however, only half of the implants showed pacing activity (Kehat et al., Nat Biotechnol. (2004) 22:1282-9; Xue et al., Circulation (2005) 111:11-20). This low success rate may be due to the heterogeneity of the transplanted cell populations.

Thus, there remains a need for the development of efficacious cell therapy to restore the pacemaker activity of a diseased heart.

### SUMMARY OF THE INVENTION

The present disclosure provides a method of detecting the presence of a sinoatrial node-like pacemaker cardiomyocyte (SANLPC) in a cell population, comprising detecting a cell that expresses one or more cardiomyocyte markers and CD34 in the cell population, wherein the detected cell is a SANLPC. The one or more cardiomyocyte markers may be selected from, for example, cTNT, SIRPA, TBX3, TBX18, SHOX2, and HCN4. In some embodiments, the SANLPC is characterized by being CD90⁻, NKX2-5⁻, and/or CD31⁻. Expression of CD34 by the SANLPC may be detected by, e.g., the presence of CD34 protein (e.g., as detected by an anti-CD34 antibody or an antigen-binding fragment thereof) or CD34 mRNA (e.g., as detected by RT-PCR). Expression of the cardiomyocyte markers also may be detected at the protein level or the RNA transcript level.

In some embodiments, the cell population is a cardiomyocyte population (e.g., human cardiomyocyte population) obtained from heart tissue (e.g., human heart tissue); or derived from PSCs (e.g., ESCs or iPSCs), multipotent stem or progenitor cells (e.g., hematopoietic stem or progenitor cells, mesodermal cells, cardiovascular (cardiac) progenitor cells), or differentiated somatic cells (e.g., fibroblast or white blood cells) through, e.g., reprogramming where needed and differentiation in vitro.

The method may further comprise determining the number of cells that are SIRPA⁺CD34⁺ and optionally CD90⁻ in the cell population.

In another aspect, the present disclosure provides a method of generating a cardiomyocyte population enriched for SANLPCs. This method comprises (a) providing a starting population of human cardiomyocytes; (b) contacting the human cardiomyocytes with an agent that binds specifically to CD34 (e.g., an anti-CD34 antibody or an antigen-binding fragment thereof); and (c) isolating cells that are bound by the agent from cells that are not bound by the agent, thereby obtaining a CD34⁺ cardiomyocyte population that is enriched for SANLPCs. The isolating step may be performed by, for example, fluorescence-activated cell sorting (FACS) or magnetic-activated cell sorting (MACS).

The SANLPCs may be characterized by being, for example, cTNT⁺, SIRPA⁺, CD90⁻, NKX2-5⁻, TBX3⁺, TBX18⁺, SHOX2⁺, HCN4⁺, and/or CD31⁻. In some embodiments, SANLPCs may be characterized by being SIRPA⁺CD90⁻CD31⁻CD34⁺.

In some embodiments, the starting population of human cardiomyocytes are obtained by culturing human pluripotent stem cells (hPSCs), such as human embryonic stem cells (ESCs) or human induced pluripotent stem cells (iPSCs), or isolated from a human heart (e.g., a donated heart). In other embodiments, the starting population of human cardiomyocytes are derived from multipotent human cells (e.g., hematopoietic stem or progenitor cells) or differentiated human cells (e.g., fibroblasts or white blood cells).

In particular embodiments, the starting population of human cardiomyocytes are obtained by a process comprising: (a) incubating hPSCs in an embryoid body medium comprising a BMP component, optionally BMP4, optionally further comprising a Rho-associated protein kinase (ROCK) inhibitor, to generate embryoid bodies; (b) incubating the embryoid bodies in a mesoderm induction medium comprising a BMP component (e.g., BMP4), an activin component (e.g., activin A), and optionally an FGF component (e.g., bFGF) to generate cardiovascular mesoderm cells.

In particular embodiments, the starting population of human cardiomyocytes are obtained by: (a) incubating mesodermal cells (e.g., cardiovascular mesoderm cells) in a cardiac induction medium comprising a Wnt inhibitor (e.g., IWP2), to generate cardiac progenitor cells; (b) culturing the cardiac progenitor cells in the absence of the Wnt inhibitor; and (c) optionally isolating cells from step (b) that are characterized by SIRPA⁺CD90⁻, thereby obtaining the starting population of cardiomyocytes. In certain embodiments, the cardiac induction medium further comprises a BMP component (e.g., BMP4), retinoic acid (RA), an activin/nodal inhibitor (e.g., SB-431542), an FGF inhibitor (e.g., PD 173074 or SU 5402), and/or VEGF.

In another aspect, the present disclosure provides a SANLPC-enriched cardiomyocyte population obtained by the methods described herein, and a pharmaceutical composition comprising the SANLPC-enriched cardiomyocyte population and a pharmaceutically acceptable carrier.

In another aspect, the present disclosure provides a pharmaceutical composition consisting of a cellular component and a carrier component, wherein the cellular component is a cell population in which more than 80% of the cells are SANLPCs, and wherein the carrier component comprises a pharmaceutically acceptable carrier. The SANLPCs may be characterized by, e.g., cTNT⁺, SIRPA⁺, CD90⁻, CD31⁻, NKX2-5⁻, TBX3⁺, TBX18⁺, SHOX2⁺, HCN4⁺, and/or CD34⁺. For example, the SANLPCs are characterized by being SIRPA⁺CD90⁻CD34⁺ and optionally CD31⁻.

In yet another aspect, the present disclosure provides a method of treating a patient in need of a cardiac pacemaker, comprising administering the present SANLPC-enriched cardiomyocyte population or pharmaceutical composition to the patient, optionally wherein the cardiomyocytes are autologous or allogeneic. The patient may have, for example, bradycardia, arrhythmia, heart failure, myocardial infarction, dyssynchrony, atrial fibrillation, left or right bundle branch block, Sick Sinus Syndrome, or congenital AV-block. Also provided are SANLPC-enriched cardiomyocyte populations and pharmaceutical compositions for use in these treatment methods, and the use of the SANLPC-enriched cardiomyocyte populations for the manufacture of a medicament to treat a patient in need of a cardiac pacemaker.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

The patent or application file contains at least one drawing executed in color. Copies of this patent or patent application publication with color drawings will be provided by the United States Patent and Trademark Office upon request and payment of requisite fees.

FIG. 1 illustrates a negative selection strategy for partially enriching SANLPCS. The figure shows representative flow cytometric analyses of NKX2-5⁺CD90⁻SIRPA⁺ cardiomyocytes and NKX2-5⁻CD90⁻SIRPA⁺ SANLPCs at day 20 of differentiation. Brightfield/GFP channel overlay images of the sorted populations are shown in the right panels. Scale bars, 200 μm. See also Protze et al., Nat Biotechnol. (2017) 35:56-68.

FIGS. 2A-E illustrate the generation and isolation of SANLPCs from non-genetically modified hPSC lines.

FIG. 2A shows the scheme of experimental protocol used to test the effect of FGF signaling on the specification of SANLPCs from day 3 cardiovascular mesoderm. BMP: BMP4. ACTA: activin A. SB: SB-431542. RA: retinoic acid.

FIG. 2B shows flow cytometric analyses of the proportion of NKX2-5⁺ cTNT⁺ cells and NKX2-5⁻cTNT⁺ SANLPCs in day 20 populations generated from mesoderm specified with either 20 ng/ml bFGF, no additional FGF (endogenous), or the small-molecule FGF inhibitor (FGFi) PD173074 (480 nM) on days 4-6 in the background of BMP4 (2.5 ng/ml), SB (5.4 μM) and RA (0.25 μM) signaling (days 3-6). The bar graph shows average proportion of NKX2-5⁺ cTNT⁺ and NKX2-5⁻cTNT⁺ cells generated in these conditions. t-test: **P<0.01 vs. NKX2-5⁻cTNT⁺ cells at endogenous (E) FGF levels; ##P<0.01 vs. NKX2-5⁺ cTNT⁺ cells at endogenous (E) FGF levels (n=5).

FIG. 2C is a pair of flow cytometric graphs showing the isolation of SIRPA⁺CD90⁻ cells from FGFi-treated populations at day 20 of differentiation to enrich for SANLPCs independent of the NKX2-5gfp/w transgenic reporter (left panel), and a flow cytometric analysis of the proportion of NKX2-5:GFP⁻cTNT⁺ SANLPCs after isolating the SIRPA⁺CD90⁻ population (right panel).

FIGS. 2D and 2E show the generation of SANLPCs from the HES2 hPSC-line. FIG. 2D shows the flow cytometric analyses of the proportion of NKX2-5⁺cTNT⁺ cells and NKX2-5⁻cTNT⁺ SANLPCs in day 20 EIES2-hPSC-derived populations generated from mesoderm specified with 480 nM FGFi on days 3-6 in the background of BMP4 (2.5 ng/ml), SB (5.4 μM) and RA (0.25 μM) signaling (days 3-6). An anti-NKX2-5 antibody was used to visualize NKX2-5⁺ cells. Bar graph shows average proportion of NKX2-5+cTNT+ and NKX2-5-cTNT+ cells generated in these conditions (n=5). FIG. 2E shows the cell sorting strategy used for isolating SIRPA-CD90⁻ population that were enriched for SANLPCs (left panel) and flow cytometric analyses of the proportion of NKX2-5⁻cTNT⁺ cells in the SIRPA⁺CD90⁻ population confirming the enrichment in SANLPCs. See also Protze, supra.

FIGS. 3A-C are graphs showing the results of single cell RNA-sequencing (scRNA-seq) of hPSC-derived SANLPC cultures. FIG. 3A shows 11 cell clusters identified at day 20 of SANLPC differentiation. FIG. 3B is a panel of t-distributed stochastic neighbor embedding (tSNE) plots showing transcript counts of the indicated genes TNNT2, NKX2-5, TBX3, TBX18, SCN5A, and NPPA. FIG. 3C is a tSNE plot showing transcript counts of CD34. CM: cardiomyocytes.

FIGS. 4A-F are graphs showing that CD34 is specifically expressed by SANLPCs. FIG. 4A shows a representative flow cytometric analysis of CD34 and NKX2-5 expression in SIRPA⁺CD90⁻ cardiomyocytes of day 20 SANLPC differentiation cultures. FIG. 4B is a bar graph summarizing the expression of CD34 in NKX2-5⁻ and NKX2-5⁺ cardiomyocytes as shown in FIG. 4A (n=5). FIG. 4C shows a representative flow cytometric analysis of the proportion of CD34⁺-CD31⁺ cells in total cells present in the day 20 SANLPC differentiation cultures (left) and in SIRPA⁺CD90⁻ gated cardiomyocytes (right) of the same culture. FIGS. 4D and E show representative flow cytometric analyses of CD34 and NKX2-5 expression in SIRPA⁺CD90⁻ cardiomyocytes of day 20 ALCM (FIG. 4D) and VLCM (FIG. 4E) differentiation cultures.

FIG. 4F is a bar graph summarizing the proportion of CD34⁺ cells within the SIRPA⁺CD90⁻ cardiomyocyte population of SANLPC, ALCM and VLCM differentiation cultures as shown in FIGS. 4A, 4D, and 4E (n=5). Error bars represent SEM. One-way ANOVA followed by Bonferroni post hoc test: **P<0.01, vs. indicated sample. ALCM: atrial-like cardiomyocytes. VLCM: ventricular-like cardiomyocytes.

FIGS. 5A-B Show that CD34⁺ cardiomyocytes can be detected as early as day 10 of differentiation. FIG. 5A shows representative flow cytometric analysis of CD34 and NKX2-5 expression in SIRPA⁺CD90⁻ cardiomyocytes from day 4 to day 20 during the SANLPC differentiation process. FIG. 5B shows a violin blot summarizing the proportion of CD34⁺ cardiomyocytes during the time course of differentiation (n=6).

FIGS. 6A-C show that FACS sorting for CD34⁺ cells enriches for SANLPCs. FIG. 6A illustrates the cell sorting strategy used for the isolation of the SIRPA⁺CD90⁻CD34⁺ and SIRPA⁺CD90⁻CD34⁻ populations in the day 20 SANLPC differentiation cultures. Samples were analyzed by flow cytometry immediately after FACS for the expression of CD34 and NKX2-5. FIG. 6B shows the proportion of NKX2-5⁻ and NKX2-5⁺ cells within the SIRPA⁺CD90⁻ myocyte population in pre-sort, CD34⁺, and CD34⁻ sorted cells (n=5). Error bars represent SEM. t-test: *P<0.05, **P<0.01. FIG. 6C shows an RT-qPCR analysis of the indicated markers for cardiomyocytes (TNNT2, NKX2-5), SAN (TBX3, SHOX2, HCN4), working cardiomyocytes and transition zone cardiomyocytes (SCNSA, NPPA) in pre-sort, CD34⁺, and CD34⁻ sorted cells (n=5). Values represent expression levels relative to housekeeping gene TBP. One-way ANOVA followed by Bonferroni post hoc test: *P<0.05, **P<0.01 vs. indicated sample.

FIGS. 7A-C show that MACS sorting for CD34⁺ cells enriches for SANLPCs. FIG. 7A illustrates the cell sorting strategy used for the MACS based isolation of the CD34⁺ and CD34⁻ populations in the day 20 SANLPC differentiation cultures. Note, MACS sorting did not include selection for SIRPA⁺CD90⁻ myocytes. Samples were analyzed by flow cytometry immediately after MACS for the expression of SIRPA and CD90 to assess the myocyte content, and CD34 and NKX2-5 to assess the pacemaker content. FIG. 7B shows the proportion of NKX2-5⁻ and NKX2-5⁺ cells within the SIRPA⁺CD90⁻ myocyte population in pre-sort, CD34⁺, and CD34⁻ sorted cells (n=5). Error bars represent SEM. t-test: **P<0.01. FIG. 7C shows an RT-qPCR analysis of the indicated markers for cardiomyocytes (TNNT2, NKX2-5), SAN (TBX3, SHOX2, HCN4), working cardiomyocytes and transition zone cardiomyocytes (NPPA) in pre-sort, CD34⁺, and CD34⁻ sorted cells (n=4). Values represent expression levels relative to housekeeping gene TBP. One-way ANOVA followed by Bonferroni post hoc test: *P<0.05, **P<0.01 vs. indicated sample.

FIGS. 8A-D show that CD34 is specifically expressed by SANLPCs generated from the HES2 hPSC line. FIG. 8A shows a representative flow cytometric analysis of CD34 expression in SIRPA⁺CD90⁻ cardiomyocytes of day 20 SANLPC differentiation cultures. The GFP channel is empty because the HES2 hPSC line does not carry the NKX2-5:GFP reporter. FIGS. 8B and C show representative flow cytometric analyses of CD34 expression in SIRPA⁺CD90⁻ cardiomyocytes of day 20 ALCM (FIG. 8B) and VLCM (FIG. 8C) differentiation cultures. FIG. 8D is a bar graph summarizing the proportion of CD34⁺ cells within the SIRPA⁺CD90⁻ cardiomyocyte population of SANLPC, ALCM and VLCM differentiation cultures as shown in FIGS. 8A-C (n=4). Error bars represent SEM. One-way ANOVA followed by Bonferroni post hoc test: **P<0.01, vs. indicated sample. ALCM: atrial-like cardiomyocytes. VLCM: ventricular-like cardiomyocytes.

FIGS. 9A-D show that MACS sorting for CD34⁺ cells enriches for SANLPCs in the HES2 hPSC line. FIG. 9A illustrates the cell sorting strategy used for the MACS based isolation of the CD34⁺ and CD34⁻ populations in the day 20 SANLPC differentiation cultures from the HES2 hPSC line. MACS sorting did not include a selection for SIRPA⁺CD90⁻ myocytes. Samples were analyzed by flow cytometry immediately after MACS for the expression of SIRPA and CD90 to assess the myocyte content, and CD34 to assess the sorting efficiency. FIG. 9B shows representative flow cytometric analysis of pre-sort, CD34⁺ and CD34⁻ sorted cells. Because the HES2 hPSC line does not express the NKX2-5:GFP reporter samples were fixed immediately after the sort and intracellular staining for the myocyte marker cTNT and NKX2-5 were performed to assess the pacemaker content. FIG. 9C shows the proportion of NKX2-5⁻ and NKX2-5⁺ cells within the cTNT⁺ myocyte population in pre-sort, CD34⁺, and CD34⁻ sorted cells (n=5). Error bars represent SEM. t-test: *P<0.05. FIG. 9D shows an RT-qPCR analysis of the indicated markers for cardiomyocytes (TNNT2, NKX2-5), SAN (TBX3, SHOX2, HCN4), working cardiomyocytes and transition zone cardiomyocytes (NPPA) in pre-sort, CD34⁺, and CD34⁻ sorted cells (n=3). Values represent expression levels relative to housekeeping gene TBP. One-way ANOVA followed by Bonferroni post hoc test: *P<0.05, **P<0.01 vs. indicated sample.

FIGS. 10A-C show single nuclei RNA sequencing (snRNA-seq) results of human SAN tissue. FIG. 10A is a tSNE plot showing 18 cell clusters identified by snRNA-seq of human fetal SAN tissue (gestation week 20). FIG. 10B is tSNE plots showing transcript counts of the indicated genes (TNNT2, NKX2-5, TBX3, TBX18, SCN5A, and NPPA). FIG. 10C is a tSNE plot showing transcript counts of CD34. Blue arrow indicates CD34 expressing cells in SAN cluster.

FIGS. 11A-B show expression of CD34 specifically in pacemaker cardiomyocytes of the human SAN. FIG. 11A shows a cross section of human fetal SAN tissue immunostained for CD34 and TBX3. Scale bar 100 μm. FIG. 11B shows a cross section of human fetal SAN tissue immunostained for CD34, NKX2-5 and cTNT. Top row, scale bar 100 μm. Bottom row, high magnification inset of yellow boxed area, scale bar 50 μm. White dotted line outlines the TBX3⁺NKX2-5⁻ SAN. DAPI was used to visualize cell nuclei.

### DETAILED DESCRIPTION OF THE INVENTION

The present disclosure provides methods of using CD34 as a cell surface marker to detect SAN-like pacemaker cardiomyocytes or cells (SANLPCs) and to generate cardiomyocyte cell populations highly enriched for SANLPCs. In some embodiments, more than 50% (e.g., more than 55%, more than 60%, more than 65%, more than 70%, more than 75%, more than 80%, more than 85%, more than 90%, more than 95%, or more than 99%) of the cardiomyocytes in the cell population are SANLPCs. As used herein, the term SANLPCs encompasses sinoatrial node pacemaker cardiomyocytes (SANPCs) obtained (e.g., isolated) from heart tissue (e.g., human heart tissue), as well as cells that have the electrophysiological functions of naturally occurring SANPCs and are generated from tissue cultures, as, for example, further described below.

The enriched populations of SANLPCs are expected to be efficacious in regenerative medicine for restoring the normal pacemaker activity and synchronized contraction of the heart chambers in a patient in need thereof (e.g., a patient with bradycardia, arrhythmia, heart failure, left or right bundle branch block, atrial fibrillation, myocardial infarction, or a certain type of congenital heart disease such as Sick Sinus Syndrome and congenital AV-block). The present enriched cell populations can be implanted, for example, into the left ventricular chamber, or directly into the SAN, AVN, the His bundle (atrioventricular bundle), or left and right bundle branch sites of a diseased heart, wherein the pacemaker cells integrate with the intact components of the patient's native cardiac conduction system and function under the control of the autonomic nervous system. These implanted cells replace the need for electronic pacemaker implantation devices and overcome the significant disadvantages of such devices.

The utility of the present SANLPC-enriched cell populations goes beyond their use as biological pacemakers. Access to enriched SANLPC is of value for in vitro studies of human SAN physiology, as well as for patient-specific disease modelling of congenital diseases affecting the SAN such as Sick Sinus Syndrome. These disease modelling studies will enable identification of potential drug targets and development of novel drug treatments. In addition, hPSC-derived SANLPCs could also be used to assess potentially dangerous off-target effects of novel drugs on pacemaker cells in safety pharmacology screens.

I. Generation of Sinoatrial Node-like Pacemaker Cells in vitro

SAN pacemaker cells are a type of cardiomyocytes. Cardiomyocytes are cells characterized by the expression of signal-regulatory protein alpha (SIRPA) and one or more of cardiac troponins (e.g., cardiac troponin I or cardiac troponin T (“cTNT”)). Cardiomyocytes are also CD90 negative. Developmentally, cardiomyocyte progenitor cells are derived from cardiac mesodermal cells (aka cardiovascular mesoderm or mesodermal cells), and are characterized by being cTNT-positive and NK2 homeobox 5 (NKX2-5)-positive. The progenitor cells differentiate into atrial and ventricular cardiomyocytes, which are NKX2-5⁺, or pacemaker cells, which are NKX2-5⁻. In further embodiments, cardiac pacemaker cells are further characterized by TBX3⁺TBX18⁺SHOX2⁺HCN4⁺NKX2-5⁻.

The spontaneous action potential generation in SAN cells is driven by the so-called funny current (If) that is encoded by HNC4 and HCN1 ion channels (Goodyer, supra). This current causes a slow diastolic depolarization until the action potential firing threshold is reached. Ventricular and atrial cardiomyocytes (also called ventricular and atrial working cardiomyocytes) do not display this spontaneous depolarization in their action potentials and are typically not spontaneous active in a mature state. Pacemaker cells and working cardiomyocytes further differ in the maximum upstroke velocity of their action potentials, which is much slower in pacemaker cells due to the low expression levels of the cardiac sodium ion channel (SCNSA) (Goodyer, supra; Liu et al., Adv Drug Deliv Rev. (2016) 96:253-73). In addition, pacemaker cells have shorter action potential durations compared to working cardiomyocytes.

A variety of cell types may be used as a source of cells for the in vitro (including ex vivo) generation of cardiomyocytes such as SANLPCs. The source cells may be, for example, human pluripotent stem cells (PSCs). As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, embryonic stem cells, PSCs derived by somatic cell nuclear transfer, and induced PSCs (iPSCs). As used herein, the term “embryonic stem cells,” “ES cells,” or “ESCs” refers to pluripotent stem cells obtained from early embryos; in some embodiments, this term refers to ES cells obtained from a previously established ES cell line and excludes stem cells obtained by recent destruction of a human embryo.

One convenient source of cells for generating cardiomyocytes such as SANLPCS is iPSCs. iPSCs are a type of pluripotent stem cell artificially generated from a non-pluripotent cell, such as an adult somatic cell or a partially differentiated cell or terminally differentiated cell (e.g., a fibroblast, a cell of hematopoietic lineage, a myocyte, a neuron, an epidermal cell, or the like), by introducing to the cell or contacting the cell with one or more reprogramming factors. Methods of producing iPSCs are known in the art and include, for example, inducing expression of one or more genes (e.g., POU5F1/OCT4 (Gene ID: 5460) in combination with, but not restricted to, SOX2 (Gene ID: 6657), KLF4 (Gene ID: 9314), c-MYC (Gene ID: 4609, NANOG (Gene ID: 79923), and/or LIN28/LIN28A (Gene ID: 79727)). Reprogramming factors may be delivered by various means (e.g., viral, non-viral, RNA, DNA, or protein delivery); alternatively, endogenous genes may be activated by using, e.g., CRISPR and other gene editing tools, to reprogram non-pluripotent cells into PSCs.

Methods of isolating and maintaining PSCs, including ESCs and iPSCs, are well known in the art. See, e.g., Thomson et al., Science (1998) 282(5391):1145-7; Hovatta et al., Human Reprod. (2003) 18(7):1404-09; Ludwig et al., Nat Methods (2006) 3:637-46; Kennedy et al., Blood (2007) 109:2679-87; Chen et al., Nat Methods (2011) 8:424-9; and Wang et al., Stem Cell Res. (2013) 11(3):1103-16.

Methods for inducing differentiation of PSCs into cells of various lineages are well known in the art. For example, numerous methods exist for differentiating PSCs into cardiomyocytes, as shown in, e.g., Kattman et al., Cell Stem Cell (2011) 8(2):228-40; Burridge et al., Nat Protocols (2014) 11(8):855-60; Burridge et al., PLoS ONE (2011) 6:e18293; Lian et al., PNAS. (2012) 109:e1848-57; Ma et al., Am J Physiol Heart Circ Physiol. (2011) 301(5):H2006-H2017; WO 2016/131137; WO 2018/098597; and U.S. Pat. No. 9,453,201.

Multipotent cells such as human mesodermal cells and cardiac progenitor cells may also be used. As used herein, a “multipotent” cell refers to a cell that is capable of giving rise to more than one cell type upon differentiation. Multipotent cells have more limited differentiation potential than pluripotent cells.

In some other embodiments, the source of cells is differentiated somatic cells that may be reprogrammed into cardiomyocyte cells. For example, the source of cells may be fibroblasts (see, e.g., Engel and Ardehali, Stem Cells Int. (2018) 2018:1-10). Direct reprogramming of fibroblasts into cardiomyocyte-like cells by overexpressing the cardiac developmental transcription factors Gata4, Mef2c, and Tbx5 (GMT) has been reported (Ieda et al., Cell. (2010) 142(3):375-86). In some embodiments, SANLPCs may then be enriched from a population of cardiomyocyte cells using methods described herein.

Developmentally, cardiomyocyte progenitor cells or cardiac progenitor cells are derived from cardiac mesodermal cells, and are characterized by being cTNT⁺. One method for generating human cardiac progenitors from hPSCs (e.g., hESCs and human iPSCs) involves (i) inducing hPSCS to differentiate into mesoderm by contacting the PSCs with a medium comprising an activator of the activin signaling pathway (e.g., an activin) and an activator of a bone morphogenetic protein 4 (BMP4) receptor (e.g., BMP4); and (ii) inducing the mesoderm to differentiate into cardiac progenitors by contacting the mesodermal cells with a Wnt signaling antagonist.

Activins are members of the transforming growth factor beta (TGF-β) family of proteins produced by many cell types throughout development. Activin A is a disulfide-linked homodimer (two beta-A chains) that binds to heteromeric complexes of a type I (Act RI-A and Act RI-B) and a type II (Act RII-A and Act RII-B) serine-threonine kinase receptor. Activins primarily signal through SMAD2/3 proteins when the activated activin receptor complex phosphorylates the receptor-associated SMAD. The resulting SMAD complex regulates a variety of functions, including cell proliferation and differentiation.

BMPs are part of the transforming growth factor beta superfamily. BMP4 binds to two different types of serine-threonine kinase receptors known as BMPR1 and BMPR2. Signal transduction via these receptors occurs via SMAD and MAP kinase pathways to effect transcription of BMP4's target genes. Various BMPs are suitable for use in generating the cells provided herein, including BMP4 and BMP2.

Wnt signaling antagonists are molecules (e.g., a chemical compound; a nucleic acid, e.g., a non-coding RNA; a polypeptide; and a nucleic acid encoding a polypeptide) that antagonize the Wnt signaling pathway, thus resulting in decreased pathway output (i.e., decreased target gene expression). For example, a Wnt signaling antagonist can function by destabilizing, decreasing the expression of, or inhibiting the function of a positive regulatory component of the pathway, or by stabilizing, enhancing the expression or function of a negative regulatory component of the pathway. Thus, a Wnt signaling antagonist can be a nucleic acid encoding one or more negative regulatory components of the pathway. A Wnt signaling antagonist can also be a small molecule or nucleic acid that stabilizes a negative regulatory component of the pathway at either the mRNA or the protein level. Likewise, a subject Wnt signaling antagonist can be a small molecule or nucleic acid inhibitor (e.g., microRNA, snRNA, etc.) of a positive regulatory component of the pathway that inhibits the component at the mRNA or protein level. In some embodiments, the Wnt signaling antagonist is a small molecule chemical compound (e.g., Xav-939, C59, ICG-001, IWR1, IWP2, IWP4, IWP-L6, pyrvinium, PKF115-584, and the like). In particular embodiments, Wnt antagonism may be achieved by the combined use of Xav-939 and C59 or the combined use of Xav-939 and IWP-L6. See also US20180251734.

For example, to generate cardiac progenitor cells, the PSCs may first be induced to aggregate to form embryoid bodies (EBs). To do so, the PSCs (e.g., hPSCs) may be cultured in an EB medium comprising a BMP component (e.g., BIVIP4), optionally further comprising a Rho-associated protein kinase (ROCK) inhibitor, for a period of time (e.g., 8-24h) to generate embryoid bodies. The EB medium may be made with a Roswell Park Memorial Institute (RPMI) base medium (optionally with B27 supplement), a Dulbecco's Modified Eagle Medium (DMEM) base medium, an Iscove's Modified Dulbecco's Media (IMDM) base medium, or StemPro®-34, with the BMP component and/or ROCK inhibitor added to it. In some embodiments, the concentration of BMP4 in the EB medium is between about 0.1 and 10 ng/ml (e.g., about 0.5-5 ng/ml, or about 0.1, 0.5, 1, 2, 3, 4, 5, 6, 7, 8, 9, or 10 ng/ml). In some embodiments, a ROCK inhibitor (e.g., Y-27632; Biotechne-Tocris #1254) in the EB medium may range in 1-20 μM (e.g., 5-15 μM such as 10 μM). For example, the EB medium may contain 1 ng/ml BMP4 and 10 μM Y-27632.

The EBs may then be cultured in a first differentiation medium (mesoderm induction medium) comprising activin A, BMP4, and optionally fibroblast growth factor-basic (bFGF; also known as basic FGF, FGF-basic, FGF-beta, FGF2, heparin binding growth factor, or FGF family members bind heparin). The mesoderm induction medium may be made with an RPMI base medium (optionally with B27 supplement), a DMEM base medium, an IMDM base medium, or StemPro®-34, with the indicated factors added to it. The selection of Activin A, BMP4, and bFGF concentrations may be based on identification of a mesoderm population that contains a high proportion ALDH⁺ cells and low proportion of CD235a⁺ cells and generates a high proportion of cTNT⁺ cells at day 20. In some embodiments, the concentration of BMP4 in the mesoderm induction medium is between about 0.1 and 30 ng/ml (e.g., about 5-10 or 5-15 ng/ml; or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In some embodiments, the mesoderm induction medium includes Activin A at a concentration of about 0.1 and 30 ng/ml (e.g., about 5-10 or 5-15 ng/ml; or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In some embodiments, the mesoderm induction medium additionally contains 0.1-30 ng/ml bFGF (e.g., about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml). In particular embodiments, the mesoderm induction medium contains about 10 ng/ml BMP4, about 6 ng/ml activin A, and about 5 ng/ml bFGF. The hPSCs may be cultured in the mesoderm induction medium for about 1-3 days (e.g., 1, 1.5, 2, 2.5, or 3 days).

After this culturing step, the cells may be further cultured for at least 1-3 days (e.g., 1, 1.5, 2, 2.5, or 3 days) in a second differentiation medium (cardiac induction medium) comprising a Wnt signaling antagonist, such as IWP2, and optionally comprising VEGF. The cardiac induction medium can be made with an RPMI base medium (optionally with B27 supplement), a DMEM base medium, an IMDM base medium, or StemPro®-34, with the indicated factors added to it. In some embodiments, the cardiac induction medium may contain IWP2 at 0.1-10 μM such as 0.5, 1, 2, 3, 4, 5, 6, 7, 8, 9, or 10 μM, and optionally VEGF at 0.1-30 ng/ml (e.g., 5-10 or 5-15 ng/ml; or 0.5, 1, 2, 3, 4, 5, 6, 7, 8, 9, 10, 11, 12, 13, 14, 15, 16, 17, 18, 19, 20, 25, or 30 ng/ml). To promote lineage commitment to pacemaker cells, the cardiac induction medium may contain one or more of BMP4, retinoic acid (RA), an activin/nodal inhibitor (e.g., SB-431542), an FGF inhibitor (e.g., PD 173074 or SU 5402), and/or VEGF. In some embodiments, the cardiac induction medium contains BMP4 at 0.5-10 ng/ml (e.g., 1-5 ng/ml or 2.5 ng/ml), RA at 0.05-2 μM (e.g., 0.1 to 0.5 μM or 0.125 μM), SB-431542 at 0.1-10 μM (e.g., 1-5 μM or 3 μM), PD 173074 at 0.05-2 μM (e.g., 0.5-1.5 μM or 1 μM).

After incubation in the cardiac induction medium, the cells may be further cultured for another one to three weeks (e.g., 7-15 days) in a base cardiac medium to obtain a cell population comprising cardiomyocytes. The base cardiac medium may be, for example, an RPMI base medium (optionally with B27 supplement), a DMEM base medium, an IMDM base medium, or StemPro®-34 that is optionally supplemented with VEGF (e.g., at 0.1-30 ng/ml as listed above).

In some embodiments, the EBs are induced to differentiate into cardiac progenitor cells (and eventually cardiomyocytes) in an EB differentiation media, commonly known as EB20 (see, e.g., Lee et al., Circ Res. (2012) 110(12):1556-63). The cardiac progenitors may then be further cultured in a base cardiac medium, such as an RPMI base medium (optionally with B27 supplement), a DMEM base medium, an IMDM base medium, or StemPro®-34, to obtain a cell population comprising human cardiomyocytes (e.g., human cardiac troponinT (cTnT⁺ cells). The base cardiac medium may contain VEGF as described above.

An alternative method for generating human cardiac progenitors from hPSCs (e.g., hPSCs and human iPSCs) involves (i) activating Wnt/β-catenin signaling in hPSCs to obtain a first cell population; and (ii) inhibiting Wnt/β-catenin signaling in the first cell population to obtain a second cell population comprising cardiomyocyte progenitors. In some embodiments, small molecules may be used to sequentially activate and inhibit Wnt/β-catenin signaling. Activation of Wnt/β-catenin signaling in hPSCs may be achieved by contacting the hPSCs with a Wnt signaling agonist. In some embodiments, a Wnt signaling agonist functions by stabilizing β-catenin, thus allowing nuclear levels of β-catenin to rise. β-catenin can be stabilized in multiple ways. As multiple negative regulatory components of the Wnt signaling pathway function by facilitating the degradation of β-catenin, a subject Wnt signaling agonist can be a small molecule or nucleic acid inhibitor (e.g., microRNA, snRNA, etc.) of a negative regulatory component of the pathway that inhibits the component at the mRNA or protein level. For example, the Wnt signaling agonist is an inhibitor of glycogen synthase kinase-3 β(GSK-3β). In some such embodiments, the inhibitor of GSK-3β is a small molecule chemical compound (e.g., CHIR-99021, TWS119, BIO, SB 216763, SB 415286, CHIR-98014, and the like). Inhibition of Wnt/β-catenin signaling may be achieved by contacting the cells that were previously contacted with the Wnt signaling agonist, with a Wnt signaling antagonist, such as those described above. In general, after ending the inhibition of Wnt/β-catenin signaling, cardiac progenitors may be further cultured in a base cardiac medium, such as an RPMI base medium (optionally with B27 supplement), a DMEM base medium, an IMDM base medium, or StemPro®-34, to obtain a cell population comprising human cardiomyocytes (e.g., human cardiac troponinT (cTnT⁺ cells).

In an exemplary, nonlimiting protocol based on the protocol described in Lian et al., supra, cardiomyocytes may be generated from human PSCs via cardiac induction using CHIR as follows. At day −1, 6E6 hPSCs may be plated and cultured on Vitronectin-coated six-well plates in E8 medium and allowed to attach to the plates overnight. At day 0, cell culture medium may be prepared by adding CHIR-99021 (“CHIR”; Tocris 4423/10) to basal cardiomyocyte (CM) medium (RPMI (with L-Glutamine)/B-27 without insulin, plus 213 μg/ml L-ascorbic acid 2-phosphate (Sigma)) to reach a CHIR concentration of 2, 4, 6, 8, 10, or 12 μM. The old medium in the plates may be replaced with 4 ml per well of CHIR-supplemented basal CM medium. Optimization of CHIR concentration may be desirable (e.g., a range of 2-12 μM CHIR may be tested). At day 1, the culture medium may be removed by aspiration. The wells may be washed once with DMEM to remove debris. Then room-temperature RPMI/B-27/without insulin medium may be added at a volume of 4 ml per well. The plates may be incubated at 37° C., 5% CO₂. At days 2 to 3, the culture medium may be removed by aspiration. The wells may be washed once with DMEM to remove debris. Then IWR1 may be added to 4 ml of fresh RPMFB-27/without insulin medium, to reach a final IWR1 concentration of 2.5 μM. At day 5, the culture medium may be replaced with room-temperature RPMI/B-27/without insulin medium at a volume of 4 ml per well. The plates may be incubated at 37° C., 5% CO₂. At days 5, 6, 7, the cell culture comprises cardiac progenitor cells. From day 7 and on, the culture medium may be replaced with room-temperature RPMI/B-27 medium at a volume of 4 ml per well to generate cardiomyocytes. The plates may be incubated at 37° C., 5% CO₂. Cardiomyocytes may be counted by flow cytometry (cTNT/NKX2-5). Robust spontaneous contraction should occur by day 12. The cells can be maintained with this spontaneously beating phenotype for more than 6 months. This protocol may be scaled up to produce large quantities of cardiac progenitors and/or cardiomyocytes. For example, bioreactors, large roller bottles, and other culturing devices may be used in lieu of multi-well tissue culture plates.

In some embodiments, the starting population is cardiovascular mesoderm cells. Such cells, express surface markers PDGRF-alpha(high) and KDR(low) (U.S. Pat. No. 10,561,687). In addition, these cells express surface marker CD56 and express MESP1 and T(Brachyury) by Q-RT-PCR, and can give rise to cTNT⁺ cardiomyocytes. Addition of FGF inhibitor to cardiovascular mesoderm cells dramatically increases the proportion of sinoatrial node-like cardiomyocytes when assessed at day 20 of culture (ibid). Accordingly, in some embodiments, the cardiovascular mesoderm cells are also treated with an FGF inhibitor for all or part of the cardiac induction phase.

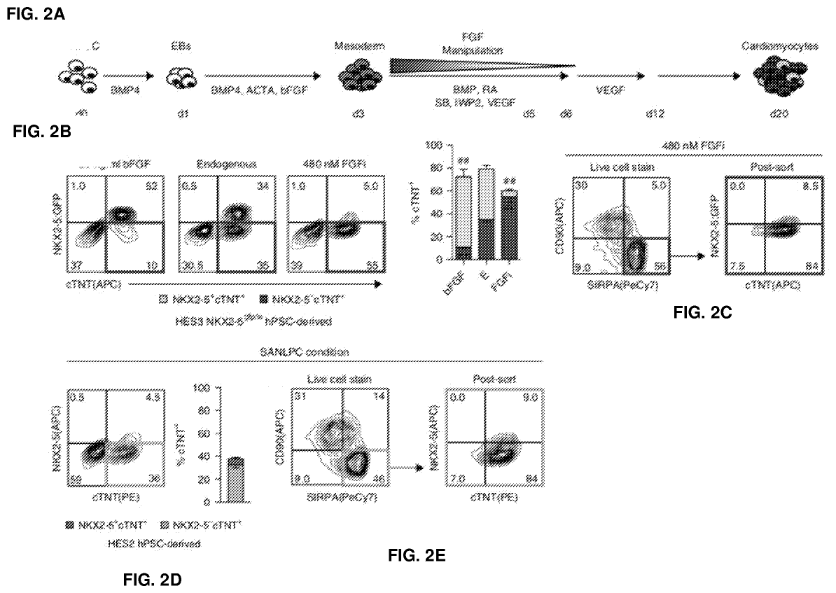

In particular embodiments, the culture methods comprise: a) incubating the hPSCs in an embryoid body medium comprising a BMP component (e.g., BMP4), optionally further comprising a Rho-associated protein kinase (ROCK) inhibitor, for a period of time to generate embryoid bodies; b) incubating the embryoid bodies in a mesoderm induction medium comprising a BMP component (e.g., BMP4), and an activin component (e.g., activin A), and optionally a FGF component (e.g., bFGF), for a period of time to generate cardiovascular mesoderm cells; c) incubating the cardiovascular mesoderm cells in a cardiac induction medium comprising a BMP component (e.g., BMP4), above a selected amount, and retinoic acid (RA), and optionally one or more of a FGF inhibitor, a Wnt inhibitor, optionally IWP2, VEGF and an activin/nodal inhibitor (e.g., SB-431542), for a period of time to generate cardiovascular progenitor cells that express TBX18 wherein the cardiovascular mesoderm cells are preferably incubated with the FGF inhibitor (e.g., PD 173074 (Tocris), SU 5402 (Tocris), and any other FGF receptor inhibitor or FGF signaling inhibitor), which FGF inhibitor is provided for all or part of a cardiac induction phase, and (d) incubating the cardiovascular progenitor cells in a basic medium comprising VEGF for a period of time to generate a population of cardiomyocytes enriched for SANLPCs. Cardiomyocytes in the culture can be isolated by using a cardiomyocyte-specific surface marker such as SIRPA and thymocyte differentiation antigen 1 (THY-1/CD90) optionally wherein the isolated population is SIRPA⁺CD90⁻. See also FIG. 2A.

The medium used at the various differentiation stages as described above can be made with any suitable base medium, which includes, without limitation, an RPMI base medium (optionally with B27 supplement), a DMEM base medium, an IMDM base medium, or StemPro®-34, with the indicated factors (e.g., cytokines and small molecules) added to the base medium.

### II. Further Enrichment of Sinoatrial Node-like Pacemaker Cells

The above-described culture methods may lead to a cardiomyocyte cell population with up to about 50% SANLPCs. However, it is desirable to obtain further enriched SANLPC cell compositions with a purity of more than about 50% (and up to about 99%), an important requirement for biological pacemaker cell therapy. The present inventors have unexpectedly discovered that cell surface molecule CD34 is a distinct marker for SANLPCs among cardiomyocytes. CD34 is a transmembrane glycoprotein typically expressed on early lymphohematopoietic stem and progenitor cells, and endothelial cells. The inventors have found that staining a population of cardiomyocytes for CD34 would allow separation of NKX2-5⁻CD90⁻SIRPA⁺ SANLPCs from other types of cardiomyocytes (See Example 2). Thus, NKX2-5⁻CD90⁻SIRPA⁺ SANLPCs can be further enriched from a population of cardiomyocytes using an agent (e.g., an antibody or an antigen-binding fragment thereof) that targets CD34 on the cell surface. Anti-CD34 antibodies are readily available from commercial sources (e.g., CD34 BV421 clone 581 and CD34 APC clone 8G12, both of BD Biosciences, Cat. Nos. 562577 and 340441, respectively). In some embodiments, contaminating endothelial cells can be removed by using CD31 binding agents such as an anti-CD31 antibody. SANLPCs are CD31⁻, while endothelial cells are CD31⁺.

Accordingly, the present disclosure provides methods of producing a population of cardiomyocytes enriched for SANLPCs by using an anti-CD34 antibody or an antigen-binding fragment thereof (e.g., scFv, scFv-Fc fusion, Fab, or F(ab′)₂), or any other agent that binds specifically to CD34 (e.g., Fc fusion proteins comprising a natural or modified ligand for CD34). Selection/enrichment of bound SANLPCs may be accomplished by using sorting methods available in the art that include, but are not limited to, fluorescence-activated cell sorting (FACS), magnetic-activated cell sorting (MACS) (e.g., immunomagnetic cell sorting), buoyancy-activated cell sorting (BACS™) (see, e.g., Lio et al., PLoS One (2015) 10(5):e0125036), and the like. Combinations of cell sorting methods may also be employed, as necessary, to reduce sort time and improve purity and recovery.

In some embodiments, the population of cardiomyocytes enriched for SANLPCs comprises at least about 50%, at least about 60%, at least about 70%, at least about 80%, at least about 90%, at least about 95%, or at least about 99% of SANLPCs. In some embodiments, the SANLPC-enriched cardiomyocyte cell population is substantially devoid of atrial-like cardiomyocytes (ALCMs) and ventricular-like cardiomyocytes (VLCMs), which are NKX2-5⁺.

Temporal expression of CD34 during the SANLPC cell culture differentiation process can be used to determine the optimal time for harvesting SANLPCs from the cell culture. For example, an aliquot of the cell culture can be assessed to measure the level of CD34 to assess whether a desired number of SANLPCs has been achieved in the cell culture. The cell culture can be assessed at one or more stages. Additional cardiomyocyte markers or SANLPC-specific markers can be measured, for example, by using immuno-based techniques (e.g., flow cytometry), or mRNA-based techniques (e.g., quantitative RT-PCR).

CD34 expression also can be used as a basis for evaluating the purity of a SANLPC composition, for example, as part of quality control for pacemaker cell therapy products. Conversely, CD34 expression can be used as a basis for detecting contaminating SANLPCs in a working cardiomyocyte (e.g., ventricular and/or atrial cardiomyocyte) composition.

### Pharmaceutical Compositions and Use

The enriched SANLPC cell preparations of the present disclosure can be used in biological pacemaker therapies to treat a subject (e.g., a human subject) with age-related loss of SAN or AVN function, a congenital disease, or a heart surgery that results in a slow, irregular heartbeat (bradyarrhythmia) with symptoms ranging from fatigue to syncope. In some embodiments, a patient in need of the present cell therapy has bradycardia, arrhythmia, left and/or right bundle branch block, atrial fibrillation, or a congenital condition such as Sick Sinus Syndrome and congenital AV-block. In some embodiments, the subject is suffering from one or more previous myocardial infarctions or heart failure. In some embodiments, the type of heart failure is a left-sided heart failure, a right-sided heart failure, a diastolic heart failure, or a systolic heart failure. In some embodiments, the heart failure is congestive heart failure. In some embodiments, patients that are indicated for pacemaker treatment include heart failure patients with reduced left ventricular ejection fraction (e.g., ≤35%), left bundle branch block (Arnold et al., J am Coll Cardiol. (2018) 72:3112-22), unsynchronized contraction of left and right ventricles (cardiac dyssynchrony; Cleland et al., N Engl J Med. (2005) 15:1539-49), and/or QRS prolongation (Bristow et al., N Engl J Med. (2004) 21:2140-50), and patients with heart failure caused by ischemic cardiomyopathies (myocardial infarction; Bristow, supra).

The SANLPC cell preparations of the present disclosure may be administered via minimally invasive methods and/or transplanted locally into a subject in need thereof. Various methods are known in the art for administering cells into a patient's heart (e.g., atrium), and include, without limitation, intracoronary administration, intramyocardial administration, or transendocardial administration. SANLPCs can be introduced to the heart by using a catheter-based approach. The catheter may be inserted via the femoral, subclavian, jugular or axillary vein, or by endocardial transplantation into the ventricle, atrium or SAN region. The cells also can be transplanted into the ventricle, atrium, or SAN region by an epicardial approach, using a needle inserted through the chest. Fluoroscopy (X-ray based method) or 3D mapping can be used to guide the catheter/needle to the intended injection site.

The SANLPC-enriched cell preparations described herein may be provided in a pharmaceutical composition containing the cells and a pharmaceutically acceptable carrier. In some embodiments, the pharmaceutical composition comprises a population of PSC-derived (e.g., iPSC-derived) SANLPCs and a pharmaceutically acceptable carrier and/or additives. For example, sterilized water, physiological saline, general buffers (e.g., phosphoric acid, citric acid, and other organic acids), stabilizers, salts, anti-oxidants, surfactants, suspension agents, isotonic agents, cell culture medium that optionally does not contain any animal-derived component, and/or preservatives may be included in the pharmaceutical composition. In some embodiments, the pharmaceutical composition is formulated into a dosage form suitable for administration to a subject in need of treatment. In some embodiments, the pharmaceutical composition is formulated into a dosage form suitable for intramyocardial administration, transendocardial administration, or intracoronary administration. For storage and transportation, the cells optionally may be cryopreserved. Prior to use, the cells may be thawed and diluted in a sterile carrier that is supportive of the cell type of interest.

A therapeutically effective number of SANLPCs are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. It will be appreciated by those of ordinary skill in the art that a therapeutically effective amount is typically administered via a dosing regimen comprising at least one-unit dose.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, cell biology, molecular described herein are those well-known and commonly used in the art. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: Differentiation of SANLPC Cardiomyocytes in Cell Cultures

This example describes protocols for differentiating SANLPCs from human pluripotent stem cells (hPSCs). The protocols also are described in Protze, supra, the entirety of which is incorporated herein by reference.

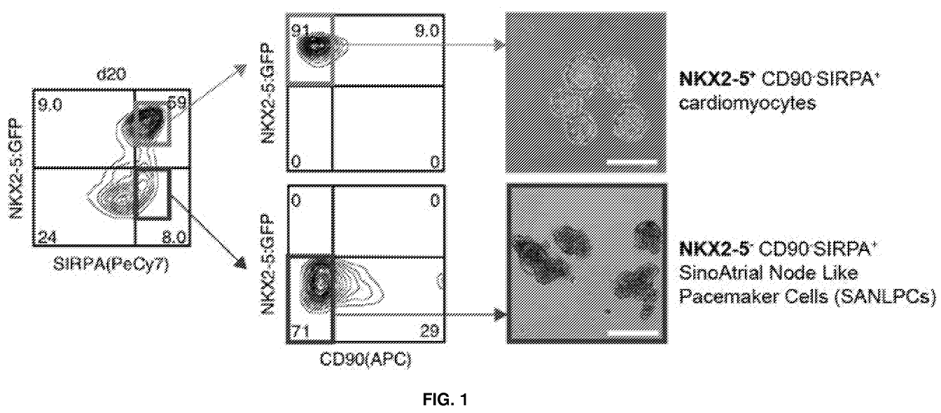

In a first protocol, SANLPCs were identified and isolated by a negative selection as NKX2-5⁻CD90⁻SIRPA⁺ cardiomyocytes using flow cytometry (FIG. 1). NKX2-5 is a cardiac transcription factor that is expressed by every cardiomyocyte except SAN pacemaker cardiomyocytes (Mommersteeg et al., Cardiovasc Res. 2010) 87:92-101; Sizarov et al., Circ Arrhythm Electrophysiol. (2011) 4:532-42). To be able to read out the expression of NKX2-5, which is an intracellular protein and not amenable for live cell staining by an antibody, a reporter human embryonic stem cell line (HES3 NKX2-5:GFP) was used; this cell line had a GFP fluorescent reporter inserted into the 1st exon of one of the NKX2-5 alleles.

Because the above protocol relies on the use of a specific reporter cell line, a second protocol was developed in which the development of any NKX2-5⁺ cardiomyocytes in the differentiation cultures was blocked by inhibiting FGF signaling during the differentiation process (FIG. 2A). In order to visualize the proportion of NKX2-5⁻ SAN pacemaker cardiomyocytes in cell lines that do not carry the NKX2-5:GFP reporter, cells were fixed and stained for intracellular NKX2-5 and cTNT protein. 2×10⁵ cells were used per staining. Briefly, EBs were dissociated using collagenase type 2 and TrypLE™. Cells were then washed and fixed with 4% paraformaldehyde in PBS(Ca²⁺Mg²⁺) for 10 min at 4° C., followed by washing twice in PBS (Ca²⁺Mg²⁺) and 5% FCS. Cells were then permeabilized in wash buffer (PBS(Ca²⁺Mg²⁺), 0.3% BSA, 0.3% Triton X, and 5% FCS) for 10 min at 4° C. Cells were then centrifuged at 3000 rpm for 4 min, resuspended, and incubated overnight at 4° C. in staining buffer (PBS(Ca²⁺Mg²⁺), 0.3% BSA, 0.3% Triton X) plus mouse anti-cardiac isoform of cTNT and rabbit anti-human NKX2-5 antibodies. Cells were then washed, centrifuged, and resuspended 2× in wash buffer. Cells were then centrifuged, resuspended, and incubated for 30 min at 4° C. in staining buffer containing goat anti-mouse PE+dk anti-rabbit Alexa Fluor 647 secondary antibodies. Cells were then washed and resuspended in wash buffer 2×. The cells were again centrifuged and resuspended in FACS buffer (PBS(Ca²⁺Mg²⁺)+5% FCS). An NKX-GFP reporter line was used as a positive control (counterstained with NKX2-5 antibody/Alexa Fluor 647).

The data obtained under the second protocol show that the resulting cultures contained non-cardiomyocytes and NKX2-5⁻ SAN pacemaker cardiomyocytes that could be isolated from the non-cardiomyocytes based on CD90⁻SIRPA⁺ expression (FIGS. 2B and 2C). Using this approach, cultures enriched in SANLPCs could be isolated from several hPSC lines (HES3, HES2, MSC-iPS1) (FIGS. 2D and 2E) (Protze, supra).

### Example 2: Identification of Cell Surface Marker for NKX2-5⁻ SANLPC Cardiomyocytes

The above protocols allow for the directed differentiation of hPSCs into SANLPCs but significant challenges remain. For applications in biological pacemaker therapies, highly enriched pacemaker populations are required. It is difficult to routinely achieve SANLPC differentiation with high efficiencies (i.e., ≥50%). For applications in disease modelling, the differentiation protocol needs to be applied to different patient-specific iPSCs. The differences in endogenous signaling between cell lines often require adjustments to the protocol, which can take four month or longer to develop in order to obtain cell cultures containing mostly NKX2-5⁻ SAN pacemaker cardiomyocytes but no NKX2-5⁺ cardiomyocytes (Protze, 2017, supra). Therefore, a cell surface marker that would allow for the positive selection of SANLPCs by fluorescence activated cell sorting (FACS) or magnetic activated cell sorting (MACS) from the differentiation cultures would be highly valuable. This cell surface marker would allow for isolation of a population highly enriched for SANLPCs (e.g., ≥80% SANLPCs) even from cardiomyocyte populations that only contain a small proportion of SANLPCs (<50%, such as no high than 5%, 10%, 20%, 30%, or 40%).

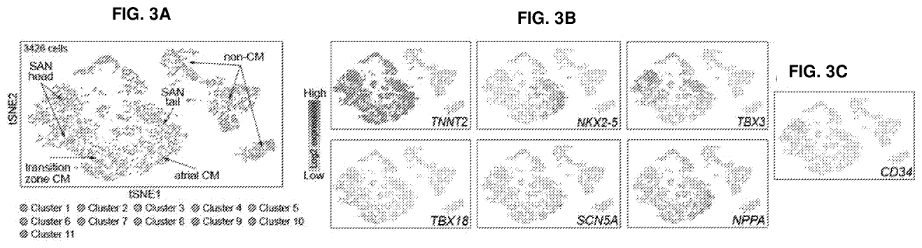

In order to identify a cell surface marker specifically expressed by NKX2-5⁻ SANLPC cardiomyocytes, we performed single cell RNA-sequencing (scRNA-seq) of hPSC-derived (HES2 hPSC line) day 20 cultures that were differentiated with the SAN pacemaker protocol described in Example 1 and in Protze, supra. The sequencing platform from 10x Genomics was used (FIG. 3A). Unsupervised clustering identified 11 distinct cell clusters, the majority of which expressed TNNT2, indicating that they represented cardiomyocytes (FIG. 3B). Clusters 1 and 2 contain cells expressing TNNT2⁺NKX2-5⁻TBX3^{high}TBX18⁺, which represented SANLPCs. We also identified three clusters of NKX2-5⁺ cardiomyocytes: TNNT2⁺NKX2-5⁺TBX3⁺TBX18⁻ that represented SAN tail cells (cluster 3), TNNT2⁺NKX2-5⁺TBX3^{low}SCN5A⁺ cells that represented transition zone cells (cluster 4), and TNNT2⁺NKX2-5⁻TBX3⁻SCN5A⁺NPPA⁺ that represented atrial cardiomyocytes (cluster 5). In an effort to identify genes that are specifically expressed by the NXK2-5⁻ SANLPCs, we performed differential gene expression analysis. Notably, this analysis revealed that the cell surface antigen CD34 was significantly enriched in clusters 1 and 2 (FIG. 3C).

To test whether staining for CD34 would mark the NKX2-5⁻ SANLPCs in the hPSC-differentiation cultures, we differentiated the HES3 NKX2-5:GFP reporter cell line using the SAN differentiation protocol described in Example 1 and Protze et al., supra. After dissociation of EBs, 1×10⁵−3×10⁵ cells were transferred to a 96 well plate, centrifuged at 2000 rpm (500 g) and resuspended in 100 μl of FACS buffer (PBS without Ca²⁺ or Mg²⁺+5% FCS+0.02% NaN₃) containing antibodies against CD34 (APC 1:300, BD Biosciences, Cat. No. 340441), SIRPA (Pe-Cy7 1:1000, BioLegend, Cat. No. 323807, clone SE5A5), and CD90 (BV421, 1:300, BD Biosciences, Cat. No. 559869). Cells were incubated for 30-45 min at 4° C. Then 100 μl of FACS buffer was added to the incubated cells. The cells were pelleted at 2000 rpm, washed in 200 μl of FACs buffer, pelleted again, and then resuspended in 200 μl of FACS buffer prior to being analyzed.

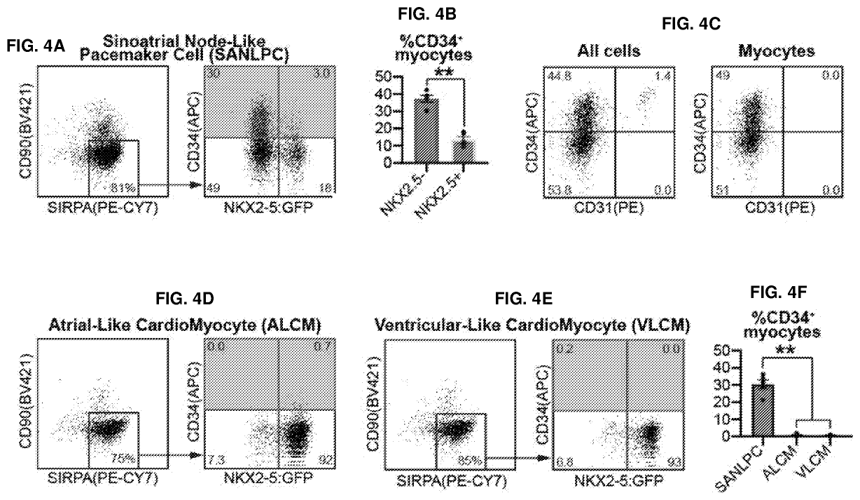

We stained the day 20 SANLPC differentiation cultures for SIRPA and the fibroblast marker CD90 to identify SIRPA⁺CD90⁻ cardiomyocytes (FIG. 4A). Including an anti-CD34 antibody (BD Biosciences, cat #340441) to this staining showed that 30±3% of the cardiomyocytes in the SANLPC differentiation cultures expressed CD34. Importantly, CD34⁺ expression was significantly enriched in the NKX2-5⁻ SANLPCs compared to the NKX2-5⁺ cardiomyocytes contained in these cultures ((37±2% vs. 13±2%) (FIG. 4B). These data were consistent with the scRNA-seq results.

Because CD34 is a known marker for endothelial cells, we also performed co-staining with the endothelial cell marker CD31 (FIG. 4C). This analysis showed that the cultures contained a small population of CD31⁺CD34⁺ endothelial cells. Importantly, the CD34⁺ cells found in the SIRPA⁺CD90⁻ cardiomyocyte population did not express CD31, confirming that those are not endothelial cells.

To further test the specificity of CD34, we next differentiated the HES3 NKX2-5: GFP reporter line into atrial (ALCM) and ventricular (VLCM) cardiomyocytes using established protocols (Lee, 2017, supra). Neither atrial nor ventricular cardiomyocytes were found to express CD34 (FIGS. 4D-F). These data suggest that CD34 specifically marks SANLPCs.

Although we also detected a population of CD34⁺CD31⁺ endothelial cells in the differentiation cultures, we do not anticipate issues with contamination from endothelial cells because the proportion of these cells was low (˜2%) (FIG. 4C).

### Example 3: Identification of SANLPCs During Time Course of Differentiation

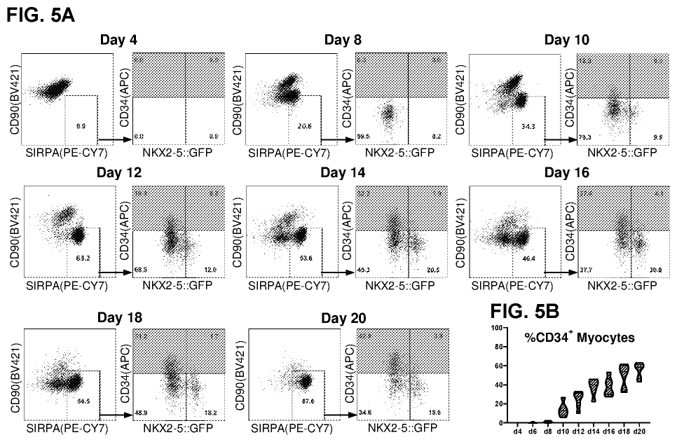

In order to test whether CD34 could be used to identify NKX2-5⁻ SANLPCs early during the SAN differentiation, we analyzed the expression of CD90, SIRPA, NKX2-5:GFP and CD34 by flow cytometry from day 4 to day 20 in the HES3 NKX2-5:GFP reporter cell line (FIG. 5). As expected, at day 4 of differentiation no SIRPA⁺CD90⁻ cardiomyocytes were detected (FIG. 5A). By day 10, a robust population of SIRPA⁺CD90⁻ cardiomyocytes was present that continued to increase until day 20. We were able to detect CD34⁺ cells within this cardiomyocyte population as early as day 10 (14±7%). The proportion of CD34⁺ myocytes continued to increase until day 20 (55±8%) (FIG. 5B). Importantly, throughout the differentiation CD34 expression was specific to the NKX2-5⁻ SANLPCs. These data suggest that CD34 can be used as early as day 10 to monitor the proportion of NKX2-5⁻ SANLPCs in SAN differentiation cultures. Being able to predict the SANLPC content early in differentiations is highly relevant for the scale up of differentiations for future cell therapy applications.

### Example 4: Enrichment of SANLPCs from SAN Differentiation Cultures

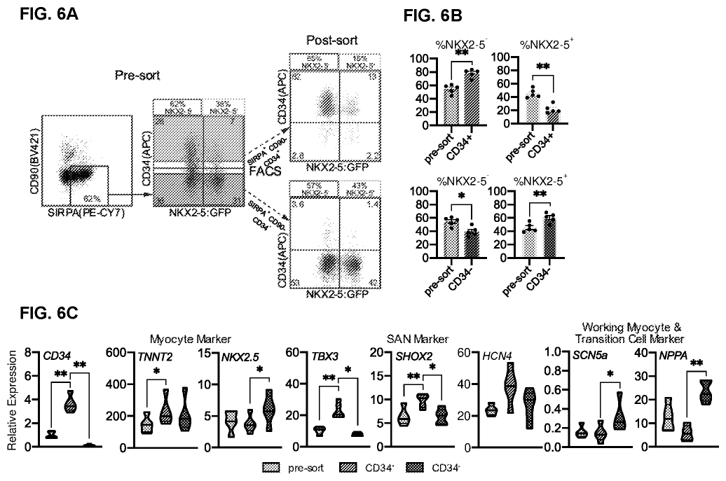

We next tested whether CD34 could be used to isolate NKX2-5⁻ SANLPCs and to separate them from the NKX2-5⁺ cardiomyocytes contained in the SAN differentiation cultures. For this study, we isolated SIRPA⁺CD90⁻CD34⁺ cardiomyocytes and SIRPA⁺CD90-CD34-cardiomyocytes from day 20 SAN differentiation cultures of the HES3 NKX2-5:GFP reporter line using FACS (FIG. 6A). We analyzed the content of NKX2-5⁻ SANLPCs immediately after the sort and found a significant enrichment of SANLPCs in the CD34⁺ sorted cells up to 78±6% compared to pre-sort control (55±7%) (FIG. 6B). Accordingly, the proportion of NKX2-5⁺ cardiomyocytes was significantly reduced in the CD34⁺ sorted cells (from 45±7% to 22±6%).

We also performed RT-qPCR analysis of the CD34⁺ and CD34⁻ sorted cardiomyocytes to test for the enrichment in SANLPC markers in the CD34⁺ sorted cells (FIG. 6C). CD34 expression was significantly enriched in the CD34⁺ sorted cells validating the sorting strategy. SIRPA⁺CD90⁻CD34⁺ sorted cells expressed higher levels of the pan-myocyte marker TNNT2 compared to pre-sort, confirming the enrichment in cardiomyocytes by selecting SIRPA⁺CD90⁻ cells. Expression of NKX2-5 was significantly reduced in the CD34⁺ population compared to the CD34⁻ population as expected for the enrichment in NKX2-5⁻ SANLPCs in the CD34⁺ population.

Consistently, the SAN pacemaker markers TBX3, SHOX2 and HCN4 were expressed at higher levels in the CD34⁺ cells than in the pre-sort and CD34⁻ cells. Expression of the sodium ion-channel gene SCN5A and the natriuretic peptide gene NPPA, which are markers for working cardiomyocytes and transition zone cells, was enriched in the CD34⁻ cells. Taken together, these data suggest that CD34⁺ specifically marks NKX2-5⁻ SANLPCs and that sorting for CD34⁺ cardiomyocytes allows for the enrichment of SANLPCs from SAN differentiation cultures. In this example, SANLPCs were enriched to a purity of 78±6%.

### Example 5: Isolation of SANLPCs Using MACS

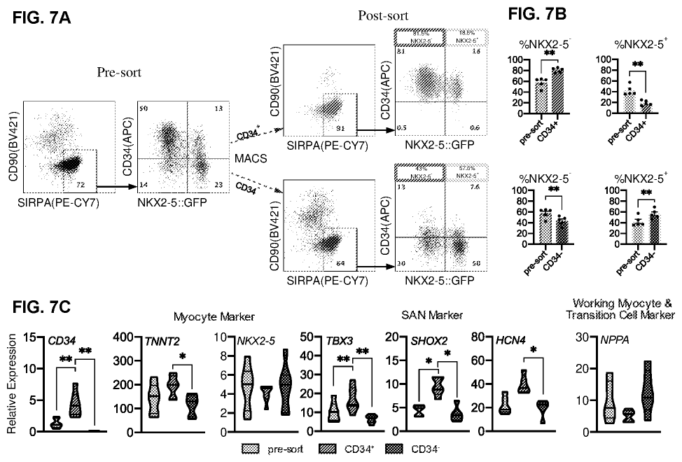

Because magnetic activated sorting (MACS) allows for the faster isolation of higher numbers of cells we tested whether SANLPCs can also be isolated using the commercially available CD34 MicroBead Kit (Miltenyi Biotec). For these experiments we used day 20 SAN differentiation cultures of the HES3 NKX2-5:GFP reporter line as described above. In contrast to the FACS based approach (Example 4) this MACS based approach does not include a selection for SIRPA⁺CD90⁻ cardiomyocytes and only separates CD34⁺ cells from CD34⁻ cells (FIG. 7A). We found that the magnetically sorted CD34⁺ cells were enriched in overall cardiomyocyte content (SIRPA⁺CD90⁻ cells) compared to pre-sort, confirming that CD34 labels cardiomyocytes contained in the differentiation cultures. In contrast, CD34⁻ sorted cells had a reduced cardiomyocyte content compared to presort. Importantly, the proportion of NKX2-5⁻ SANLPCs within the SIRPA⁺CD90⁻ cardiomyocyte population was significantly enriched up to 81±4% in the CD34⁺ sorted cells compared to pre-sort control (57±4%) (FIG. 7B). Accordingly, the proportion of NKX2-5⁺ cardiomyocytes was significantly reduced in the CD34⁺ sorted cells (from 42±9% to 19±4%).

RT-qPCR analysis of the MACS sorted CD34⁺ and CD34⁻ cells showed significantly higher CD34 expression in the CD34⁺ sorted cells and confirmed the sorting strategy (FIG. 7C). Expression of the pan-myocyte marker TNNT2 was significantly higher in CD34⁺ cells compared to CD34⁻ cells, which is in agreement with the flow cytometry results (FIG. 7A) and further confirms the higher overall cardiomyocyte content in CD34⁺ sorted populations. Expression of NKX2-5 was reduced in the CD34⁺ cells as expected based on the reduction in the proportion of NKX2-5⁺ cells seen by flow cytometry (FIG. 7B). The SAN pacemaker markers TBX3, SHOX2 and HCN4 were expressed at significantly higher levels in CD34⁺ cells than in pre-sort or CD34⁻ cells. Expression of the working cardiomyocytes and transition zone cell marker NPPA was reduced in CD34⁺ cells compared to pre-sort and CD34⁻ cells. In summary, these data are comparable to the results we obtained using FACS sorting for SIRPA⁺CD90⁻CD34⁺ cells and demonstrate that MACS sorting with CD34 MicroBeads is a viable option to enrich for NKX2-5⁻ SANLPCs to a purity of 81±4%. Using this MACS based approach will enable to isolate large numbers of SANLPCs that will be required in future transplantation experiments to assess their biological pacemaker capacity in animal models.

### Example 6: Isolation of SANLPCs from Other hPSC Cell Lines Using CD34

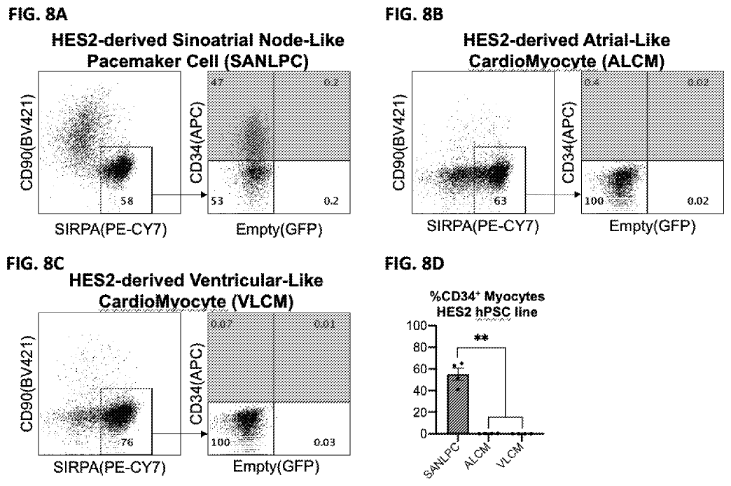

To demonstrate that CD34 can be used as a cell surface marker to isolate NKX2-5⁻ SANLPCs from any hPSC line we also applied this approach to the HES2 human embryonic stem cell line. We applied the SAN differentiation protocol to the HES2 cell line and analyzed cultures for the expression of CD34 at day 20 of differentiation. The same protocol as described in Example 2 was used to stain the cells for flow cytometric analysis. Using the anti-CD34 antibody (BD Biosciences, cat #340441) showed that 55±11% of the SIRPA⁺CD90⁻ cardiomyocytes in the SANLPC differentiation cultures expressed CD34 (FIGS. 8A and 8D).

Importantly, we also differentiated this HES2 cell line into atrial (ALCM) and ventricular (VLCM) cardiomyocytes using established protocols (Lee, 2017, supra). Similar to the results in the HES3 NKX2-5:GFP reporter line, neither atrial nor ventricular cardiomyocytes did express CD34 (FIGS. 8B-D). These data suggest that CD34 also specifically marks SANLPCs derived from the HES2 cell line.

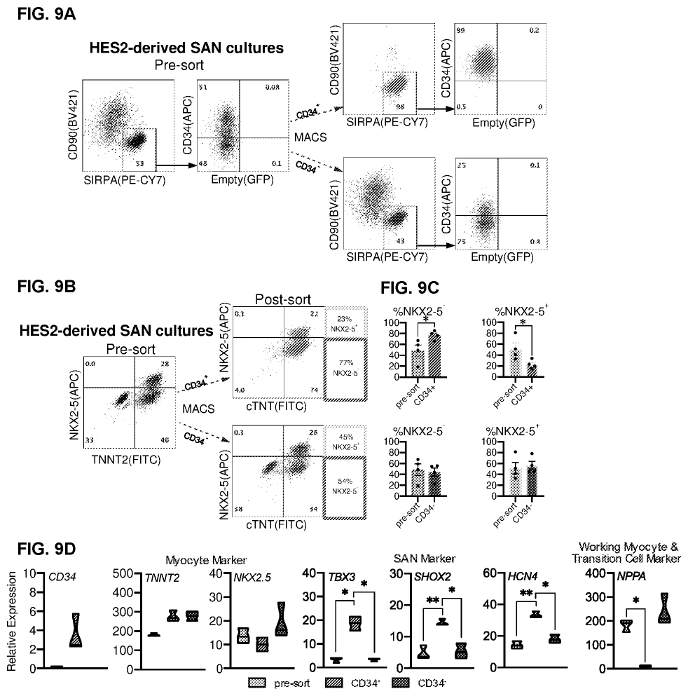

In order to test if CD34 can be used to isolate NKX2-5⁻ SANLPCs and to separate them from NKX2-5⁺ cardiomyocytes contained in the SAN differentiation cultures of the HES2 cell line we MACS sorted CD34⁺ and CD34⁻ cells as described in Example 5. As seen for the HES3 NKX2-5:GFP reporter line magnetically sorted CD34⁺ cells were enriched in overall cardiomyocyte content (SIRPA⁺CD90⁻ cells) while CD34⁻ sorted cells had a reduced cardiomyocyte content compared to presort (FIG. 9A). Because the HES2 cell line does not have the NKX2-5:GFP reporter we used intracellular antibody staining for NKX2-5 (1:1000, Cell Signaling, Cat. No. 8792S, clone E1Y8H) and the myocyte marker cTNT (1:2000, Thermo Scientific, Cat. No. MS-295-P, clone 13-11) in fixed cells to assess the NKX2-5 expression in the sorted cells (FIG. 9B). This analysis showed that the proportion of NKX2-5⁻ SANLPCs within the cTNT⁺ cardiomyocyte population was significantly enriched up to 77±4% in CD34⁺ sorted cells compared to pre-sort control (48±11%) (FIG. 9C). Accordingly, the proportion of NKX2-5⁺ cardiomyocytes was significantly reduced in the CD34⁺ sorted cells (from 52±11% to 23±4%).

RT-qPCR analysis of the MACS sorted CD34⁺ and CD34⁻ cells showed higher CD34 expression in the CD34⁺ sorted cells and confirmed the sorting strategy (FIG. 9D). Expression of the pan-myocyte marker TNNT2 was higher in CD34⁺ cells compared to pre-sort cells, which is in agreement with the flow cytometry results (FIGS. 9A and 9B). Expression of NKX2-5 was reduced in the CD34⁺ cells as expected based on the reduction in the proportion of NKX2-5⁺ cells seen by flow cytometry (FIGS. 9B and 9C).

The SAN pacemaker markers TBX3, SHOX2 and HCN4 were expressed at significantly higher levels in CD34⁺ cells than in pre-sort and CD34⁻ cells. Expression of the working cardiomyocytes and transition zone cell marker NPPA was significantly reduced in CD34⁺ cells compared to pre-sort. Taken together, this data demonstrates that MACS for CD34⁺ cells successfully enriches in NKX2-5⁻ SANLPCs from the HES2 cell line to a purity of 77±4%. Accordingly, it is expected that this approach can be extended to any other hPSC line.

### Example 7: Expression of CD34 in Human SAN Cardiomyocytes

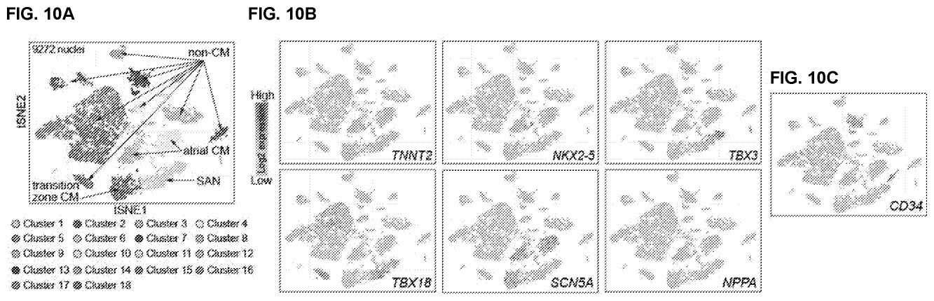

To validate that CD34 is a true SAN marker beyond the hPSC culture system, we next tested whether CD34 is expressed by SAN cardiomyocytes of the human heart. We used a previously described protocol to isolate cell nuclei from frozen heart tissue and performed single nuclei RNA-seq (snRNA-seq) (Selewa et al., Scientific Reports (2020) 10:1535; and Wu et al., J Am Soc Nephrol. (2019) 30:23-32). SAN tissue was dissected from a fetal heart (gestation week 19) and 1×10⁶ nuclei were isolated. The nuclei were then subject to sequencing using a 10x Genomics system (FIG. 10A).

Unsupervised clustering identified 18 distinct cell clusters. The TNNT2 expressing cell clusters represent cardiomyocytes. A TNNT2⁺NKX2-5-TBX3^{high}TBX18⁺ cell cluster was identified, indicating that we successfully isolated SAN cell nuclei (cluster 1) (FIG. 10B). In addition, we identified a TNNT2⁺NKX2-5⁺TBX3^{mid}SCN5A⁺ transition zone cell cluster (cluster 2) and TNNT2⁺NKX2-5⁺TBX3⁻SCN5A⁺NPPA⁺ atrial cardiomyocyte clusters (cluster 3 and 4) similar to the cardiomyocyte clusters we identified in the in vitro cultures described above. When we analyzed CD34 expression in the human heart scRNA-seq sample, high levels of CD34 expression were identified in endothelial cell clusters (cluster 12, 13, 14) (FIG. 10C). CD34 was also expressed in the fibroblast cell clusters (cluster 16, 17, 18). Importantly, CD34 expression was significantly enriched in the SAN cluster (cluster 1, blue arrow, adjusted P value 1.97 e-62) when comparing the expression to the other cardiomyocyte clusters (atrial cardiomyocytes and transition zone cardiomyocytes; clusters 2, 3, 4).

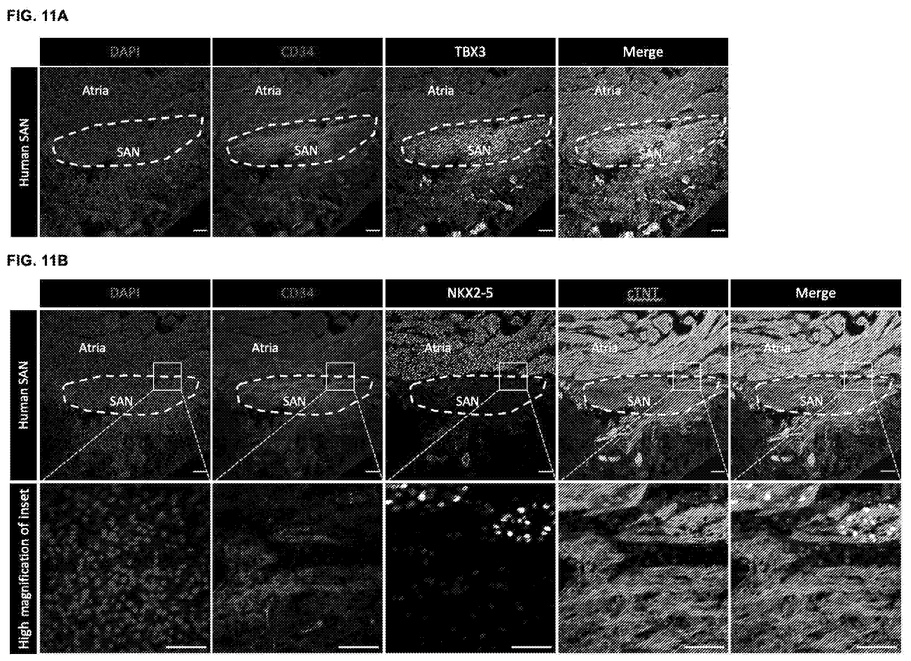

To confirm CD34 expression in the pacemaker cardiomyocytes of the human SAN on the protein level we dissected SAN tissue from a human fetal heart (gestation week 19), PFA fixed it and embedded it in paraffin to prepare cross sections. Sections were immunostained with a TBX3 antibody (1:1000, ThermoFisher, Cat. No. 424800, polyclonal) and a NKX2-5 antibody (1:1000, Cell Signaling, Cat. No. 8792S, clone E1Y8H) to identify the TBX3⁺NKX2-5⁻ pacemaker cardiomyocytes of the SAN. This approach allowed to identify a TBX3⁺ SAN area (outlined by white dotted line) next to TBX3⁻ atrial tissue (FIG. 11A). Importantly, this SAN area stained positive for CD34 (1:100, BD Biosciences, Cat. No. 340441). Staining of a consecutive section for NKX2-5 allowed to identify NKX2-5⁻ SAN cells in the same area that are distinct from the NKX2-5⁺ atrial cells (FIG. 11B). These NKX2-5⁻ SAN cells stained positive for CD34. The cardiomyocyte marker cTNT (1:100, Miltenyi, Cat. No. 130-119-575, clone REA400) was used as counterstain to confirm that the NKX2-5⁻CD34⁺ cells represent cardiomyocytes. Importantly, the TNNT2⁺NKX2-5⁺TBX3⁻ atrial cardiomyocytes did not stain positive for CD34. The CD34⁺ cells found within atrial tissue were TNNT2⁻ and most likely represent endothelial cells of blood vessels.

Taken together, these data suggest that CD34 is specifically expressed by SAN pacemaker cardiomyocytes but not by atrial cardiomyocytes in the human heart.

1. A method of detecting the presence of a sinoatrial node-like pacemaker cardiomyocyte (SANLPC) in a cell population, comprising detecting a cell that expresses one or more cardiomyocyte markers and CD34 in the cell population, wherein the detected cell is an SANLPC.

2. The method of claim 1, wherein the one or more cardiomyocyte markers are selected from cTNT, SIRPA, TBX3, TBX18, SHOX2, and HCN4, optionally wherein the SANLPC is characterized by being CD90−, NKX2-5−, and/or CD31−.

3. The method of claim 1, wherein expression of CD34 by the SANLPC is detected by the presence of CD34 protein or CD34 mRNA, optionally wherein the CD34 protein is detected by an anti-CD34 antibody or an antigen-binding fragment thereof, or the CD34 mRNA is detected by RT-PCR.

4. The method of claim 1, wherein the cell population is a cardiomyocyte population, optionally obtained from heart tissue, optionally human heart tissue, or derived from pluripotent stem cells, multipotent stem or progenitor cells, or differentiated somatic cells, optionally wherein
the pluripotent stem cells are embryonic stem cells or induced pluripotent stem cells,
the multipotent stem or progenitor cells are hematopoietic cells, or
the differentiated somatic cells are fibroblasts.

5. The method of claim 1, further comprising determining the number of cells that are SIRPA+CD34+ and optionally CD90− and/or CD31− in the cell population.

6. A method of generating a cardiomyocyte population enriched for sinoatrial node-like pacemaker cardiomyocytes (SANLPCs), comprising:
(a) providing a starting population of human cardiomyocytes;
(b) contacting the human cardiomyocytes with an agent that binds specifically to CD34; and
(c) isolating cells that are bound by the agent from cells that are not bound by the agent, thereby obtaining a CD34+ cardiomyocyte population that is enriched for SANLPCs.

7. The method of claim 6, wherein the agent is an anti-CD34 antibody or an antigen-binding fragment thereof.

8. The method of claim 6, wherein the isolating step is performed by fluorescence-activated cell sorting or magnetic-activated cell sorting.

9. The method of claim 6, wherein the SANLPCs are characterized by being CD34⁺ and further being cTNT⁺, SIRPA⁺, CD90⁻, NKX2-5⁻, TBX3⁺, TBX18⁺, SHOX2⁺, HCN4⁺, and/or CD31⁻.

10. The method of claim 6, wherein the starting population of human cardiomyocytes are obtained by culturing human pluripotent stem cells (hPSCs), optionally wherein the hPSCs are human embryonic stem cells (ESCs) or human induced pluripotent stem cells (iPSCs).

11. The method of claim 6, wherein the starting population of human cardiomyocytes are derived from multipotent human cells or differentiated human cells in vitro, optionally wherein
the multipotent human cells are hematopoietic stem or progenitor cells, or
the differentiated human cells are fibroblasts or white blood cells.

12. The method of claim 6, wherein the starting population of human cardiomyocytes are obtained by a process comprising:
(a) incubating hPSCs in an embryoid body medium comprising a BMP component, optionally BMP4, optionally further comprising a Rho-associated protein kinase (ROCK) inhibitor, to generate embryoid bodies;
(b) incubating the embryoid bodies in a mesoderm induction medium comprising
a BMP component, optionally BMP4,
an activin component, optionally activin A, and
optionally an FGF component, optionally bFGF,
to generate cardiovascular mesoderm cells.

13. The method of claim 6, wherein the starting population of human cardiomyocytes are obtained by:
(a) incubating mesodermal cells in a cardiac induction medium comprising a Wnt inhibitor, optionally IWP2, to generate cardiovascular progenitor cells, optionally wherein the mesodermal cells are cardiovascular mesoderm cells;
(b) culturing the cardiovascular progenitor cells in the absence of the Wnt inhibitor; and
(c) optionally isolating cells from step (b) that are characterized by SIRPA+CD90−,
thereby obtaining the starting population of cardiomyocytes.

14. The method of claim 13, wherein the cardiac induction medium further comprises
a BMP component, optionally BMP4,
retinoic acid,
an activin/nodal inhibitor, optionally SB-431542,
an FGF inhibitor, optionally PD 173074 or SU 5402, and/or
VEGF.

15. The method of claim 6, wherein the starting population of human cardiomyocytes are obtained from a human heart.

16. A SANLPC-enriched cardiomyocyte population obtained by the method of claim 6.

17. A pharmaceutical composition comprising the cardiomyocyte population of claim 16 and a pharmaceutically acceptable carrier.

18. A pharmaceutical composition consisting of a cellular component and a carrier component, wherein the cellular component is a cell population in which more than 80% of the cells are SANLPCs, and wherein the carrier component comprises a pharmaceutically acceptable carrier.

19. The pharmaceutical composition of claim 18, wherein the SANLPCs are characterized by cTNT+, SIRPA+, CD90−, CD31−, NKX2-5−, TBX3+, TBX18+, SHOX2+, HCN4+, and/or CD34+.

20. A method of treating a patient in need of a cardiac pacemaker, comprising administering the cardiomyocyte population of claim 16 or a pharmaceutical composition consisting of a cellular component and a carrier component, wherein the cellular component is a cell population in which more than 80% of the cells are SANLPCs, and wherein the carrier component comprises a pharmaceutically acceptable carrier to the patient, optionally wherein the cardiomyocytes are autologous or allogeneic.

21. (canceled)

22. (canceled)

23. The method of claim 20, the cardiomyocyte population wherein the patient has bradycardia, arrhythmia, heart failure, myocardial infarction, dyssynchrony, atrial fibrillation, left or right bundle branch block, Sick Sinus Syndrome, or congenital AV-block.
